(19) United States
(12) Patent Application Publication
(10) Pub. No.: US 2005/0113843 A1
(43) Pub. Date: May 26, 2005

(54) Remotely actuated system for bone cement delivery

(76) Inventor: Yves P. Arramon, Capitola, CA (US)

Correspondence Address: BOZICEVIC, FIELD & FRANCIS LLP, 1900 UNIVERSITY AVENUE, SUITE 200, EAST PALO ALTO, CA, 94303, US

(21) Appl. No.: 10/723,248

(22) Filed: Nov. 25, 2003

(51) Int. Cl. A61B 17/58

(52) U.S. Cl. 606/94; 600/432; 604/131; 604/187

(57) ABSTRACT

An injection system for delivering viscous fluids is disclosed. Exemplary fluids included flowable hard tissue implant material such as Polymethylmethacrylate (PMMA). The system comprises a manual actuator, a control line and a dispenser. The control line may be flexible cable set within a housing running between the actuator and dispenser head. In which case, the actuator is designed to enable reciprocal movement of the cable relative to the cable housing. The actuator may be designed for one-handed use like a syringe. Components of the dispenser may be utilized as a mixing vessel prior to inserting a drive unit to enable fluid delivery. Prior to use, fluid is loaded into a portion section of the drive unit from a reservoir section of the dispenser head by retracing the cable via the retractor. Fluid is expelled from the dispenser portion of the system upon advancing the cable.

### FIELD OF THE INVENTION

This invention relates to apparatus for flowing discrete amounts of material to a hard tissue implantation site. More particularly, it may involve systems for flowing bone cement to effect vertebroplasty.

### BACKGROUND OF THE INVENTION

The mineralized tissue of the bones of the human skeletal system are generally categorized into two morphological groups: “cortical” bone and “cancellous” bone. The outer walls of all bones are composed of cortical or “compact” bone. This tissue is characterized by a dense structure with only microscopic porosity. Cancellous or “trabecular” bone is found in the interior of bones. This tissue is composed of a lattice of interconnected slender rods and plates called “trabeculae.”

Injectable Polymethylmethacrylate (PMMA), set within the trabeculae, has been used for supplementing cancellous bone, especially for anterior and posterior stabilization of the spine in metastatic disease. See, Sundaresan, et al., “Treatment of neoplastic epidural cord compression by vertebral body resection and stabilization,” J Neurosurg 1985;63:676-684; Harrington, “Anterior decompression and stabilization of the spine as a treatment for vertebral collapse and spinal cord compression from metastatic malignancy.” Clinical Orthodpaedics and Related Research 1988;233:177-197; and Cybulski, “Methods of surgical stabilization for metastatic disease of the spine.” Neurosurgery 1989;25:240-252. Deramond et al., “Percutaneous vertebroplasty with methyl-methacrylate: technique, method, results [abstract],” Radiology 1990; 117 (suppl): 352, among others, have described the percutaneous injection of PMMA into vertebral compression fractures by the transpedicular or paravertebral approach under CT and/or fluoroscopic guidance.

Percutaneous vertebroplasty is desirable from the standpoint that it is minimally invasive as compared to the alternative of surgically exposing a hard tissue site to be supplemented with PMMA or other filler. Several procedures are known for accessing a desired site in the cancellous bone of a vertebral body (or for that matter other cancellous bone) to deliver hard tissue implant material to stabilize—or build up—a site once expanded as taught by U.S. Pat. Nos. 6,280,456; 6,248,110; 5,108,404 and 4,969,888.

To gain access to a hard tissue implantation site, as described in U.S. Pat. No. 6,019,776 and 6,033,411, a straight needle or cannula in combination with a stylet may be employed. U.S. patent application Ser. No. 10/366,992, entitled, “Bone Access System”, discloses another means of access. Once access is achieved, hard tissue implant material is delivered through the delivery device. The manner in which it is delivered may depend on the application and/or the capacities of the hardware employed. The invention described may be used in any setting or with any approach as apparent to those with skill in the art, including as applicable to the above-referenced writings.

In any case, numerous high pressure applicators have been developed for delivering bone cement. Those of note employ a threaded interface to generate adequate pressure to drive implant material as it becomes increasingly viscous. Each of the systems offered by Parallax Medical, Stryker, Cardinal Medical/IFlow, and Spinal Specialties are setup to deliver sufficient material to effect vertebroplasty (i.e., they are configured to deliver between about 9 cc and 12 cc) from a chamber where a piston drives the cement therefrom.

Such an approach works very well. Yet, it engenders certain disadvantages that the present invention seeks to avoid. Particularly, to effect vertebroplasty each pressure applicator is used in conjunction with a delivery conduit. The purpose of the conduit is to allow an operator to remove his or her hands some distance from the imaging field, while torquing the components of the applicator to generate internal pressure. The use of a conduit prevents transmission of movement involved in actuating the device to an emplaced delivery cannula.

Unfortunately, providing a conduit of any substantial length (which has a sufficiently small cross section so its volume does not unacceptably deplete the reservoir of bone cement of the injector) means accounting for a significant pressure drop between the entrance of the conduit to its exit.

The need to account for such requirements, in large part, for the need to generate significant pressure within know applicators. Another factor contributing to the same is the manner in which many bone cements (e.g., PMMA) begin to cure immediately upon preparation, thereby becoming more viscous during a medical procedure.

The present invention offers an alternate solution to the challenges faced with existing hardware and approaches. This alternate solution offers certain advantages as will be apparent in review of the following. Yet, it is to be appreciated that certain variations of the invention may possess every such advantage—while in others the it may not be the case.

### SUMMARY OF THE INVENTION

The present invention concerns a driver for flowable material that may be actuated by way of a remotely located physical connection. The pressure driver is suited for effecting vertebroplasty in that it includes various adaptations suited to such procedures. Optional adaptations of this variety include a closed-container mixing system that interfits with the base of the pressure driver to feed material to a pump. In which case, a feed system is provided that enables filling the drive chamber of the device with material for delivery which isolates the mixing chamber during fluid delivery. In this manner, system compliance as a result of any air or void within the mixing chamber is avoided.

The delivery device preferably utilizes a piston that is advanced and/or retracted by means of remote link. Such means may include: a cable set within a housing or a fluid column. The driver configuration will vary depending on the nature of the system.

Whatever the case, the present invention includes systems comprising any of the features described herein. Methodology described in association with the devices disclosed also forms part of the invention. Such methodology may include that associated with completing a vertebroplasty procedure and use of such auxiliary equipment as described below or otherwise available. The invention may be used in other methods as well. For instance, the invention further comprises such hardware and methodology as may be used in connection with that described which is incorporated by reference.

### BRIEF DESCRIPTION OF THE DRAWINGS

Each of the figures diagrammatically illustrates aspects of the invention. Of these:

FIG. 1 is a side view of a known high pressure injection system;

FIG. 2 is a side view of an remotely actuatable injection system according to the present invention;

FIGS. 3A and 3B detail manipulation of the actuator;

FIGS. 4A and 4B provide side, sectional views of an alternate driver used in accordance with the present invention;

FIGS. 5A and 5B provide such views of an alternative driver;

FIG. 6 is a side sectional view of the driver container of the device shown in FIGS. 5A and 5B;

FIG. 7 shows the manner in which the container in FIG. 6 is put to use; and

FIG. 8 is partial perspective view showing yet another system according to the present invention.

Variation of the invention from that shown in the figures is contemplated.

### DETAILED DESCRIPTION OF THE INVENTION

Before the present invention is described in detail, it is to be understood that this invention is not limited to particular variations set forth and may, of course, vary. Various changes may be made to the invention described and equivalents may be substituted without departing from the true spirit and scope of the invention. In addition, many modifications may be made to adapt a particular situation, material, composition of matter, process, process act(s) or step(s), to the objective(s), spirit or scope of the present invention. All such modifications are intended to be within the scope of the claims made herein.

Methods recited herein may be carried out in any order of the recited events which is logically possible, as well as the recited order of events. Furthermore, where a range of values is provided, it is understood that every intervening value, between the upper and lower limit of that range and any other stated or intervening value in that stated range is encompassed within the invention. Also, it is contemplated that any optional feature of the inventive variations described may be set forth and claimed independently, or in combination with any one or more of the features described herein.

All existing subject matter mentioned herein (e.g., publications, patents, patent applications and hardware) is incorporated by reference herein in its entirety except insofar as the subject matter may conflict with that of the present invention (in which case what is present herein shall prevail). The referenced items are provided solely for their disclosure prior to the filing date of the present application. Nothing herein is to be construed as an admission that the present invention is not entitled to antedate such material by virtue of prior invention.

Reference to a singular item, includes the possibility that there are plural of the same items present. More specifically, as used herein and in the appended claims, the singular forms “a,” “and,” “said” and “the” include plural referents unless the context clearly dictates otherwise. It is further noted that the claims may be drafted to exclude any optional element. As such, this statement is intended to serve as antecedent basis for use of such exclusive terminology as “solely,” “only” and the like in connection with the recitation of claim elements, or use of a “negative” limitation. Unless defined otherwise herein, all technical and scientific terms used herein have the same meaning as commonly understood by one of ordinary skill in the art to which this invention belongs.

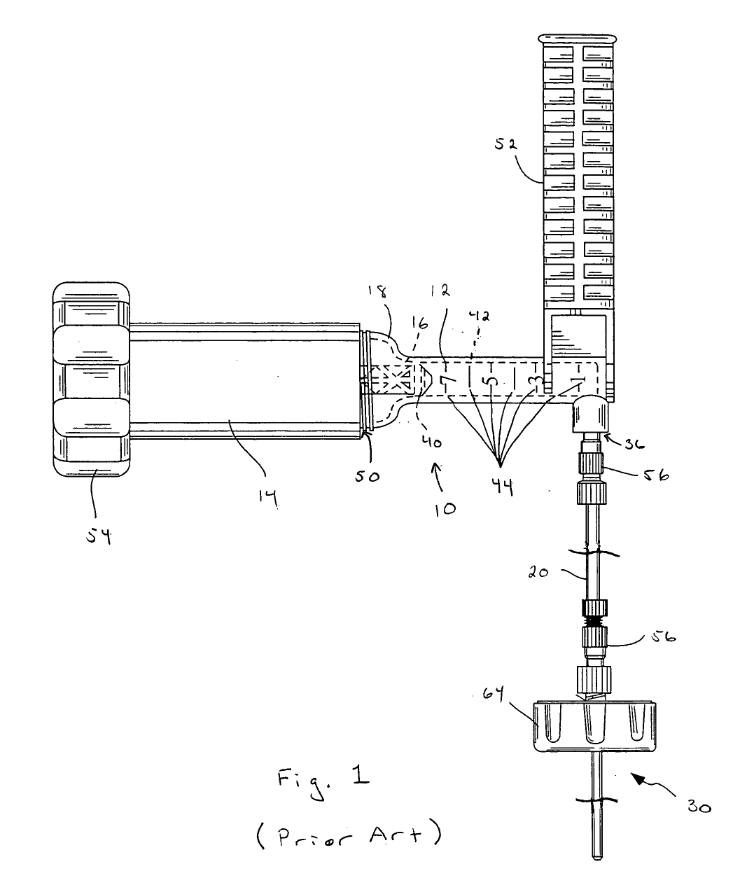

Turning now to FIG. 1, a known system suitable for use in vertebroplasty or another hard tissue implantation procedure is shown. This system has been commercialized by the present Assignee and is the subject of U.S. patent application Ser. No. 09/408,690 entitled, “High Pressure Delivery System.” The system includes a high pressure applicator 10 comprising first and second columns 12, 14 a conduit 20 for connection to the applicator 10 and a cannula 30.

A plunger or piston 16 is sized to freely pass into an introduction section 18 of the first column to avoid trapping air bubbles. The piston, by way of an O-ring 40 forms a pressure seal within bore of vessel section 42. In use, vessel section 42 is filled with bone cement. By overfilling the vessel section slightly into the introduction section, introduction of air bubbles into the vessel section where a seal capable of driving implant material is avoided.

For use in vertebroplasty and other bone augmentation procedures, PMMA implant material is preferably used. Compositions as described in U.S. Pat. Nos. 6,232,615 and 6,309,420, each incorporated herein by reference, including contrast agent and particles to facilitate tracking the progress of injected material in-process may be employed employed. Gradations 44 provide an indication of the amount of implant material within the pressure applicator.

To drive implant material from the device, plunger 16 is driven into vessel section 42. Such action is accomplished via a threaded interface 50 between the columns. Generally, radially extending handle 52 is held stationary while knob 54 is turned.

As alluded to, a non-compliant tube or conduit 20 is generally provided as a means of conveying material between the pressure applicator and an emplaced cannula 30. The components may be connected via luer fitting 52 or otherwise. Specific details regarding and advantages of utilizing a non-compliant delivery conduit are set forth in U.S. Pat. No. 6,348,055.

Essentially, the conduit is useful in allowing one to position the applicator in a comfortable position for torquing the handles relative to each other at some distance away from the imaging field. Still further, being flexible, the conduit does not transmit too much movement from the applicator to the cannula 30. Yet, as noted above, the practical length of the conduit is limited. Basically, it is thought not to be feasible or practical to make it much longer 18 or 24 (at most 36) inches in length due to device pressure generation limiting and burst strength limitations, and various other pertinent parameters such as loss of deliverable volume of implant material.

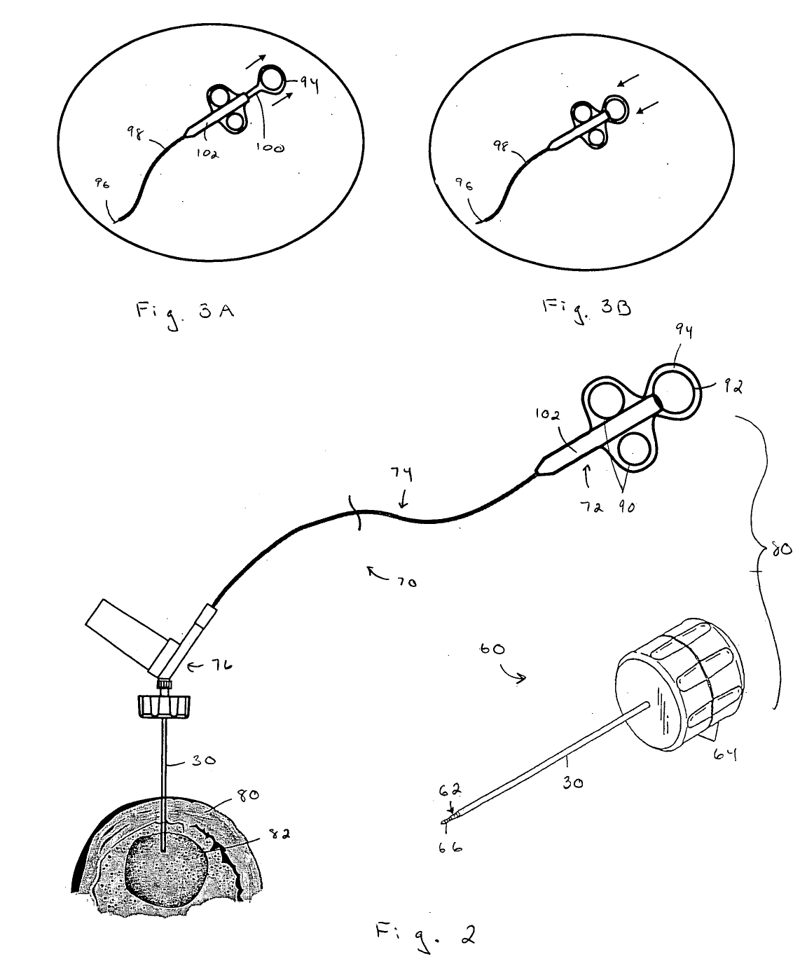

As provided in further detail in FIG. 2, cannula 30 may be provided as part of a cannula/stylet or a cannula/obturator combination 60 includes a cannula with handle portions 44 and a tubular body 48. FIG. 2 shows combination 60 with the inventive system 70 in order that it may be appreciated that such components may be provided in packaged combination in order to provide a kit 80 as one variation of the invention.

As for the cannula/stylet combination, however, both stylet 62 (received within the cannula) and cannula 30 are shown to include an optional handle portion 64. The stylet shown includes threads 66 at a terminal end. Such a tip or another sort such as a single-beveled tip, conical tip, diamond-shaped tip or some other may be provided as known in the art to assist in traversing hard and/or soft tissue to reach an implantation site. Other suitable stylet configurations are described in U.S. Pat. No. 6,033,411 and U.S. patent application Ser. No. 09/409,948, filed Sep. 30, 1999, each entitled, “Precision Depth Guided Instruments For Use In Vertebroplasty,” as well as U.S. patent application Ser. No. 09/876,387, entitled, “Cannula System of Hard Tissue Implant Delivery,” filed Jun. 6, 2001, all of which are incorporated herein by reference, may be employed.

With such tools adapted for precutaneous bone access, a surgeon initially identifies a landmark with the aid of fluoroscopy of other imaging technique. Next, an injection is given to anesthetize the skin where insertion will occur. Local anesthesia will typically also be administered to the target site as well. After sufficient time has passed to effectively anesthetize the skin, an incision is made through the skin with a scalpel. A combined stylet/cannula combination is then inserted through the incision and advanced using a translation motion with no torquing, until the tip of the stylet abuts the hard bone tissue to be traversed. Once contact has been made, the cannula tube is then grasped with a pair of hemostats and fluoroscopy/imaging is used to assess the position of the cannula/stylet with regards to the vertebra. The hemostats are used to allow the hands of the user to be removed form the field in which the imaging radiation will be applied. With the aid of medical imaging (possibly applied along various trajectories), the cannula/stylet are positioned with the desired orientation for passing into the body of the bone.

If the advancement of the stylet and cannula does not proceed along the intended pathway, the stylet may be reversed rotated while preventing rotation of the cannula to maintain it in position and remove the stylet. Then, a beveled tip stylet may be employed. With the beveled tip, the operator can rotate the sytlet to position the tip in a direction toward which he/she wishes to migrate the stylet.

Once the orientation of the stylet and cannula (the latter having been advanced over the stylet), has been satisfactorily set, the fluoroscopy/imaging is discontinued, the hemoststs are removed and the operator carefully gasps the cannula/stylet being careful not to alter the orientation. The stylet with beveled tip is then removed and replaced by the stylet with self-tapping threads. Grasping the combination handle, and optionally the cannula tube, the operator then proceeds to both push translationally and torque the combination handle to begin the threading the stylet end into hard bone tissue.

After “biting” into the bone with a few turns of the self-tapping threads, the operator's hands are removed and the devices maintain their position by the support provided by the bone surrounding the threads. The devices/instruments are again viewed fluoroscopically or otherwise imaged both along the longitudinal axis of the cannula/stylet and laterally to determined the depth of the instruments. If the desired depth and placement has not yet been achieved, imaging is discontinued, and the cannula/stylet are further torqued or otherwise advanced into the cancellous bone until the tip of the cannula has been positioned in the desirable location.

Upon achieving the desired placement of the cannula at a site for treatment, the operator withdraws the stylet from the cannula, leaving the bore of the cannula open. The cannula at this state is effectively press-fit into the bone site which aids the operator in preventing its rotation. Once the stylet has been completely removed for the cannula, fluoroscopic imaging/viewing of the cannula may optionally be performed to assure that the cannula did not move during the removal of the stylet.

Optionally, a contrast agent, e.g. a product known as OMNIPAQUE 300 available from Nycomed in Princeton, N.J., may be injected through the cannula and the flow of the contrast agent is viewed fluoroscopically or with other imaging in order to ascertain that the tip of the cannula has not been placed in a vein or other significant vessel. Preferably, the contrast agent is injected through tubing connected to the cannula. When tubing is used, it is preferably of a smaller length and diameter than tubing that is used for injection of implant material. Contrast agent must be flushed out of the site prior to injection of the implantation material, so it is preferable to inject only a small volume of the contrast agent. Viewing of the flow of the contrast agent helps to identify the shape of the body into which the injection of implant material is to be performed, as well as to locate where the major veins lie. After completing the flow of the contrast agent, the remnants of the contrast agent are flushed by injecting a flushing solution (e.g. saline) through the cannula tube, using a syringe or other injector. The imaging is preferably discontinued for this step. The contrast agent is flushed out so that it does not occlude, cloud, or otherwise compete with the viewing of the radiopacity of the implant material when it is placed.

FIG. 2 shows a cannula 30 so-emplaced. Note, however, that some other procedure or approach may be followed in accordance with the invention. In this regard, it is specifically contemplated that system 70 might further be used in connection with the Bone Access System noted above to effect hard tissue implantation at a radially remote site. Likewise, the site to which it is intended to deliver material may vary. Accordingly, body 80 may be a vertebral body, the end of a long bone or any other structure. In any case, the comments of system 70 and its manner of operation may be the same.

Specifically, the variation of the delivery system shown in FIG. 2 includes/comprises an actuator 72, a control line 74 and a pump 76. The details, especially of the pump section are elaborated upon below. But first, the basic operation of system 70 is discussed.

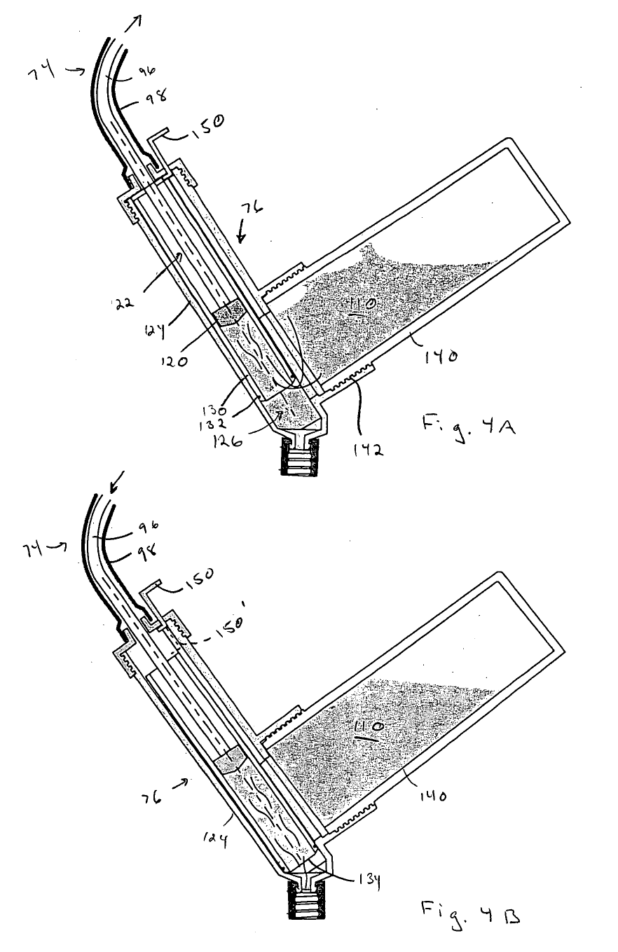

Namely, FIGS. 3A and 3B show the manner in which the system is advantageously actuated. Actuator 72 is designed with finger holes 90 and thumbhole 92 to facilitate one-handed operation. With features provided thus (though other grip configurations are possible), a user can easily manipulate the device to effect the activity shown in FIGS. 3A and 3B. In FIG. 3A, ring 94 is withdrawn, pulling on cable 96 received within cable housing 98. Typically, a rod or another sort of linking member 100 is provided and adapted to be slidingly received within a body 102 of the actuator. The cable may be connected to the rod in any conventional manner.

However configured, the purpose of withdrawing cable 96 is to prime or fill a drive chamber within pump 76. Upon compression of the actuator pushing the cable forward into the pump section as shown in FIG. 3B, implant material 82 is forced into the implant site.

As shown, this occurs directly through cannula 30. Yet, depending on the pump configuration and how much pressure it can generate an intermediate flexible conduit may be employed. Still, the preferred mode of operation is depicted. In a preferred mode, the cannula is between about 5 and about 6 inches long. With a 13 gauge cannula, implanting PMMA in a vertebral site, in this manner only between about 10 and about 120 psi of pressure need be generated within the delivery device for a reasonable delivery rate (i.e., between about 0.1 cc/sec up to about 1 cc/sec, preferably about 0.5 cc/sec requiring roughly 60 psi pressure generation within the piston bore.)

The length of the cable (or such other actuation connection as may be provided between the actuator 72 and the pressure diver or pump 76) may vary. It is possible that the cable may be very short (e.g., on the order of 2 to 6 inches). Such a setup would still offer value in that the connection between the pump and actuator would be such that unwanted movement is not transmitted to the cannula by way of actuating the pump.

However, longer length control lines 74 are preferred. They offer greater flexibility in actuator positioning and, moreover, enable a user to further remove him or herself from the imaging field: even providing a comfortable position for a physician holding the device in front of his or her abdomen while viewing the x-ray or imaging monitor in a standard operating room setup, or conveniently placing a radiation shield in between the doctor and patient. Any additional distance from the radiation field is of great benefit since the radiation intensity drops off by the inverse square of the distance (i.e., 1/r²). Accordingly, to offer a benefit over known systems control line is preferably about a foot long, about 36 inches, more preferably about 48 inches or more.

In generating the desired drive pressures, the cross-sectional are of the pump bore may be between about 0.125 and about 0.25, more preferably about 0.185 (a diameter similar to that of a typical 1 cc syringe), and still be effectively driven in a one-handed manner as shown. Should a larger actuator be desired in which greater force may be directed down the control line, then a larger diameter pump chamber may be employed without loss in pressure generation capabilities. Also, whereas the actuator shown has a travel or throw of between about 1 and about 3.5 inches another style of actuator may be employed.

Each of these aforementioned parameters will determine the volume that may be delivered by pump 76 in each cycle. Accordingly, the volume of implant material delivered in each shot may range from about 0.2 cc to about 2 cc or more. It will especially be the case that higher volume capacity (larger diameter pump chamber) may be enjoyed with implant materials that are generally less viscous and/or do not become more viscous during a procedure as does PMMA.

Regardless, FIG. 2 should suggest to one with skill in the art that several shots of implant material have been delivered through cannula 30 to form the bolus of material 82 depicted. The number of cycles will depend on the nature of the space to be filled and delivery capacity of the pump.

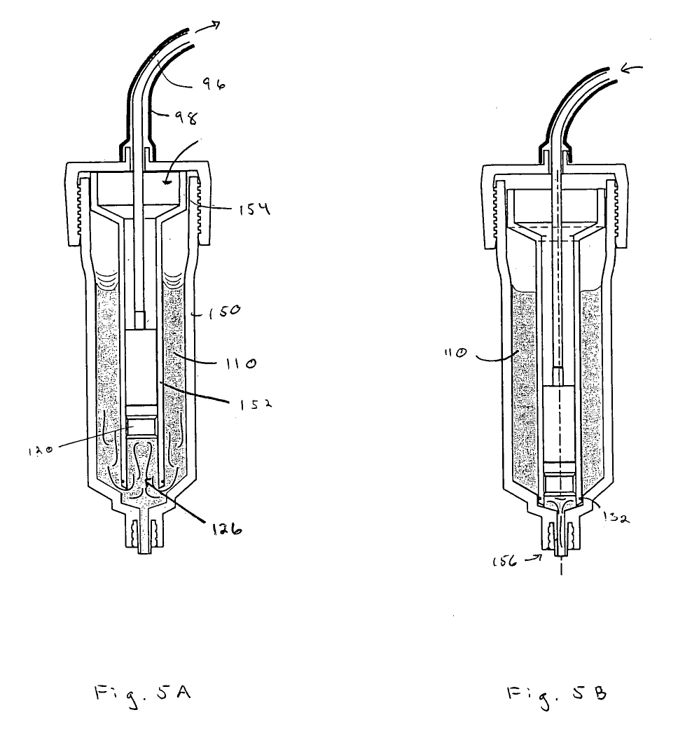

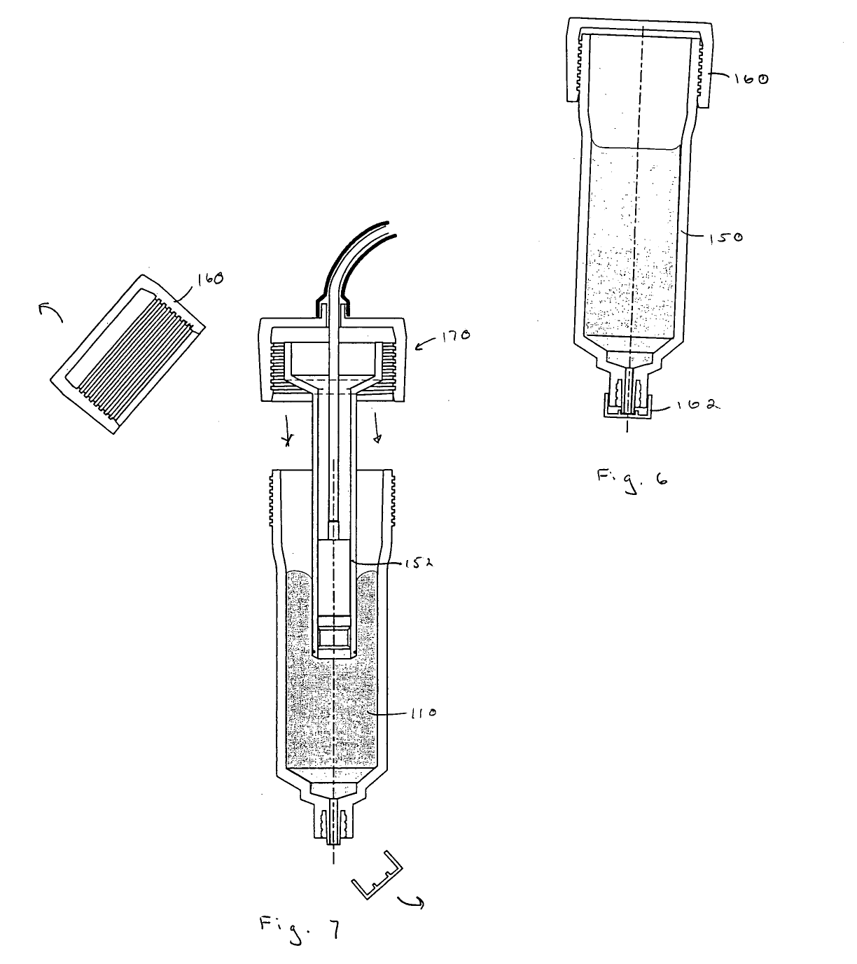

As illustrated in FIGS. 4A-5B, the pump is such that it draws fluid from a reservoir 110. The pump or pressure driver detailed in FIGS. 4A and 4B differs in configuration from that in FIGS. 5A and 5B. Yet, their principle load/expel/reload functionality is identical. Still, they vary with respect to some useful details that will be noted below.

Regarding their basic operation, however, each of FIGS. 4A and 5A show what occurs in the pumps upon taking action like that in FIG. 3A. Likewise, 4B and 5B show what occurs in the pumps upon taking action like that in FIG. 3B.

Specifically, in FIGS. 4A and 4B, the pump 76 configuration shown in FIG. 2 is provided in a cross-sectional view. Pictured thus, retraction and advancement of cable 96 within housing 98 is seen successively in the figures. A piston head 120 is shown connected directly to cable 96. However, an intermediate interface member, such as a rod which is less prone to bend or buckle upon compression, in an open zone 122 of pump 76 may be desired. Of course, this “open zone” will expand as the piston is pushed forward. Likewise, it may be completely filled if the piston is retraced fully within a barrel 124 of the pump. Often, it will be the case that the length of the barrel and corresponding available travel for the piston will be set to correspond to a full stroke for the actuator 72.

One reason for such configuration is that it minimizes the amount of implant material present in drive chamber 126. Accordingly, negative effects associated with material compressibility are minimized. Primary among these is a springiness to the system, from which rebound can occur that continues to drive the delivery of implant material after that which is intended by the controller.

FIGS. 4A show the drive chamber open and being filled with implant material; FIG. 4B shows the drive chamber closed-off from the reservoir 110 of implant material. Such action is accomplished by sleeve 130, with which the piston forms a pressure-tight seal. At least partially because of this seal, the sleeve is drawn upward to open a flowpath to the reservoir upon withdrawing the piston head.

By virtue of a seal between the body/barrel section 124 of the pressure driver and a container 140 in which implant material 110 resides, the implant material is drawn into the expanding drive chamber section as illustrated. While a threaded interface 140 between the container and barrel elements is shown, other means of securing and forming a pressure tight seal between the members may be employed. For example, various clamping, sliding or other arrangements may be employed.

In any case, upon advancing the piston (whether after a partial actuator stroke, or a full one), sleeve 130 advances as well. A seal member 132 such as an O-ring may be provided to form or enhance 134 the manner in which a distal end 134 of the sleeve closes off the barrel section from the container section to prevent backflow thereto—thus ensuring intended delivery of implant material. Of course, other sealing approaches may be employed as well, such as conical-shaped sections that push into each other or yet other approaches.

Regardless, FIGS. 4A and 4B illustrate another optional component as may be employed. Particularly, a locking mechanism 150 may be provided to lock sleeve 130 in a “reservoir closed” position. Doing so as indicated by callout 150′, allows withdrawal of implant material that had previously been injected. After setting the lock, an operator would merely need to withdraw the control cable (or such other mean provided to actuate the piston head) and the implant material is drawn back into the drive chamber.

The embodiments of the invention in FIGS. 4A and 4B are advantageous in that the easily accommodate such a lock. The embodiments of the invention in FIGS. 5A and 5B are shown without a lock. Yet, one might be incorporated therein.

In any case, these variations of the invention differ from those discussed above in that container 150 serves both as the pump body and offers reservoir 110 space. In this case, sleeve 152 slides within container 150 guided at a proximal end by wall 154. Of course, additional guide members may be provided distally.

However configured in this regard, as shown in FIG. 5A, withdrawing the piston and the sleeve together allows priming the drive chamber. Then, as shown in FIG. 5B, advancing the piston forms a seal with the container, closing off the reservoir of implant material and driving it out of the device.

The material is shown driven from a luer-lock style nozzle 156. Of course, other connector configurations may be utilized as well—such as shown in FIGS. 4A and 4B.

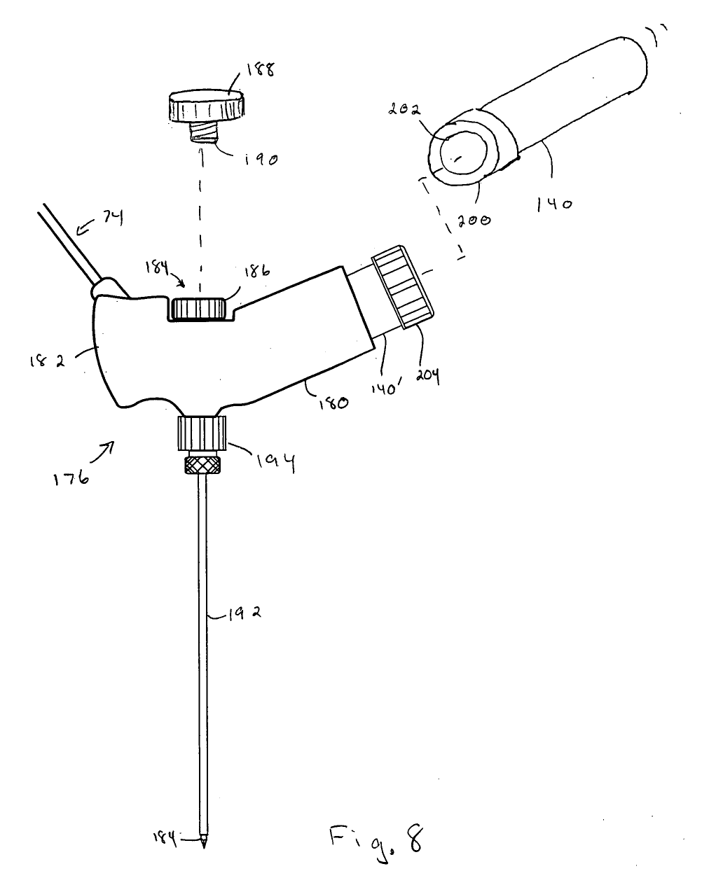

In any case, FIGS. 6 and 7 better illustrate the manner in which the system shown in FIGS. 5A and 5B is put to use. Specifically, FIG. 6 shows container 150 alone. A threaded cap 160 is provided at one end, and a press-fit cap 162 at the other. Generally, there will be no need for a user to remove cap 162 until the container is ready for use. However, cap 160 provides access to container 150 in order that one or more of the component parts of the implant material may be placed therein. With the constituents of (e.g., bone cement, any radiopaque or radiographic material) in place and the cap returned, the entire assembly can be shaken or otherwise agitated to mix the material as warranted.

Next, as shown in FIG. 7, cap 160 is discarded. Assembly 170, including a cap-type interface member 172 to which cable housing 98 is connected and the piston and sleeve, is substituted therefore. With cap 162 also discarded, the assembled system is as shown in FIGS. 5A and 5B. To avoid trapping air adjacent the piston, is may be advanced beyond sleeve prior to or during introduction into implant material reservoir 110.

The variation of the invention in FIG. 8 offers additional optional features. Namely, the driver 176 is provided in the form of a handle with first and second arms 180, 182 to provide leverage to a cannula/style combination. Stylet 184 includes a knob top 186. Upon emplacement of the cannula (aided by any of various tip features at the end of the stylet—such as treading, point, etc.), the stylet is removed from the driver to allow implant material delivery through cannula 188 once a plug 190 is inserted as shown. The plug may include an O-ring to enhance or form a seal with the driver body. Alternatively, or additionally, a valve or internal seal may be provided to close-off the space left open by removing the stylet.

Regardless, cannula 192 may be provided integral with the handle, or it may be secured thereto at an interface or hub section 194. In this manner, an off-the-shelf cannula may be used. Another optional feature is to configure the handle section(s) with a flattened top to facilitate use with a mallet.

Arm 182 contains the driver piston and sleeve features like those shown in FIGS. 4A and 4B. Arm 180 is adapted to interface with a container 140. The container may be used both to hold implant material and for mixing the same. However used, container 140 is advantageously provided with a cap 200 that can be pierced by a complimentary element within handle arm 180 to access implant material within the container. The perforable or piercable nature of the cap may be provided by a foil insert 202, or otherwise. Even if a back or secondary cap 204 is provided in another container configuration 140′, such an embodiment will preferably have a breachable end at the other side provided in some manner in order to access the mixed implant material therein.

The various components of the system will typically be made of conventional materials. For example, the stylet/cannula members from stainless steel, the container and driver, actuator and driver components from injection molded plastic such as nylon, etc. Any further constructional details regarding the invention are believed to be within the grasp of those with an ordinary level of skill in the art to which this invention pertains.

1. An implant material injection system, the system comprising:
a pressure driver, and a separate container for implant material, wherein said driver and said separate container are adapted to form a sealed pressure-tight interface between each other,
said pressure driver comprising a piston and a sleeve, wherein said piston and said sleeve are adapted to draw implant material into at least a portion of a chamber defined by said sleeve upon retracting said piston and to expel implant material from said pressure drive at a pressure level upon advancing said piston.

2. The system of claim 1, wherein said pressure level reaches at least 10 psi.

3. The system of claim 1, wherein said pressure level does not exceed 120 psi.

4. The system of claim 1, further comprising a means for remote actuation.

5. The system of claim 1, further comprising a remote actuator connected to said pressure driver.

6. The system of claim 5, wherein said remote actuator comprises first and second grip portions and is adapted to drive said piston by reciprocal movement of said grip portions relative to each other.

7. The system of claim 6, wherein said movement of said actuator corresponds 1 to 1 with movement of said piston.

8. The system of claim 7, wherein a cable set within a housing connects said actuator and said pressure driver.

9. The system of claim 8, wherein said cable is about 36 inches in length or more

10. The system of claim 9, wherein said cable is about 48 inches long.

11. An implant material injection system, the system comprising:
a pressure driver, a container for implant material, and a remote actuator connected to said pressure driver,
said pressure driver comprising a piston and a sleeve, wherein said piston and said sleeve are adapted to draw implant material into at least a portion of a chamber defined by said sleeve upon retracting said piston and to expel implant material from said pressure drive at a pressure level upon advancing said piston,
said remote actuator comprising first and second grip portions and being adapted to drive said piston by reciprocal movement of said grip portions relative to each other.

12. The system of claim 11, wherein said movement of said actuator corresponds 1 to 1 with movement of said piston.

13. The system of claim 12, wherein a cable set within a housing connects said actuator and said pressure driver.

14. The system of claim 11, wherein a remote connection between said remote actuator and said pressure drivers is about 36 inches or more long.

15. The system of claim 11, wherein said pressure level reaches at least about 10 psi.

16. The system of claim 11, wherein said pressure level does not exceed about 120 psi.

17. An implant material injection system, the system comprising:
a pressure driver, a container for implant material, and a means for remote actuation of said pressure driver,
said pressure driver comprising a piston and a sleeve, wherein said piston and said sleeve are adapted to draw implant material into at least a portion of a chamber defined by said sleeve upon retracting said piston and to expel implant material from said pressure drive at a pressure level upon advancing said piston.

18. A method of delivering flowable implant material, the method comprising:
providing an implant material injection system comprising a pressure driver and container for implant material;
connecting said pressure driver to a cannula emplaced at a location for implant material delivery;
loading implant material into said pressure driver;
driving material from said pressure driver into said implant material location;
again loading implant material into said pressure driver; and
again driving material from said pressure driver into said implant material location.

19. The method of claim 1, wherein said implant material injection system further comprises a remote actuator connected to said pressure driver, and the method further comprises:
withdrawing a first portion of said actuator relative to a second portion of said actuator to effect said loading; and
advancing said first portion toward said second portion to effect said driving.

20. The method of claim 19, wherein aid withdrawing and said advancing is performed at about 36 or greater from said location for implant material.

21. The method of claim 19, wherein a cable within a housing is provided to connect said remote actuator to said pressure driver.

22. The method of claim 19, wherein a pressure level for driving said implant material reaches at least about 10 psi.

23. The method of claim 19, wherein a pressure level for driving said implant material does not exceed about 120 psi.
